(19) United States
(12) Patent Application Publication
(10) Pub. No.: US 2021/0353804 A1
(43) Pub. Date: Nov. 18, 2021

(54) LARGE AREA AUTONOMOUS DISINFECTANT/DECONTAMINATION VEHICLE

(71) Applicant: Pratt & Miller Engineering and Fabrication, Inc., New Hudson, MI (US)

(72) Inventors: Simon DEAN, West Bloomfield, MI (US); Chris ANDREWS, Northville, MI (US); Gary LATHAM, Whitmore Lake, MI (US)

(21) Appl. No.: 17/320,715

(22) Filed: May 14, 2021

Related U.S. Application Data
(60) Provisional application No. 63/024,778, filed on May 14, 2020.

(51) Int. Cl. A61L 2/22; B05B 5/08; G05D 1/02

(52) U.S. Cl. CPC A61L 2/22; B05B 5/085; G05D 1/0238; A61L 2202/14; G05D 1/0255; G05D 1/0257; A61L 2202/16; G05D 1/0219

(57) ABSTRACT

An autonomous vehicle for applying a disinfecting/decontamination solution to surfaces within buildings, in outdoor spaces or other structures. The autonomous vehicle uses a dispensing system having an electrostatic charging mechanism which causes atomized dispensing fluid to be attracted to surfaces desired to be disinfected or decontaminated.

### RELATED APPLICATIONS

This application claims benefit of U.S. Application No. 63/024,778, filed May 14, 2020, the entire contents of which are incorporated herein by reference in its entirety.

### FIELD OF THE INVENTION

This invention relates to an autonomous disinfectant/decontamination vehicle system capable of disinfecting surfaces in buildings and other areas in an autonomous manner.

### BACKGROUND

There is a need for disinfecting surfaces in an effort to combat the spread of diseases including recently the Covid-19 virus. For example, such disinfecting may be necessary or desired in areas like airports, factories, workplaces, stadiums, downtown areas, shopping malls and districts, city parks and many others.

Disinfecting agents are used on an increasing occurrence to assure the safety of transportation passages through manual cleaning and manual spray applications. For large areas, daily application of disinfecting agents by manual application is often conducted using human spraying resources doing such work in a non-automated manner.

By using an autonomous vehicle to deliver a disinfecting agent, reduced costs can be realized for large area disinfection. In addition, such approaches offer increase reliability and repeatability, 24/7 availability, and reduced exposure to humans of pathogens and disinfecting agents. Moreover, by automating the process a validation of disinfection can be provided.

Numerous types of disinfecting agents may be applied to surfaces. For example, alcohol-based liquid solutions can be applied via fabric cloth or aerosol application. Technologies including UV light, ozone generation and others may also be used. In embodiments of the present invention, electrostatic spraying of atomized liquid disinfecting agents is used. Electrostatic spraying offers the benefits of more fully coating of surfaces as compared with fogging, traditional spraying and UV light solutions that can only disinfect “line of sight” presented surfaces. Electrostatic charging of the spray offers the ability to “wraparound” surfaces for more complete application of disinfecting materials.

### BRIEF DESCRIPTION OF THE DRAWINGS

FIG. 1 is a pictorial view of an autonomous vehicle in accordance with the present invention.

### DETAILED DESCRIPTION

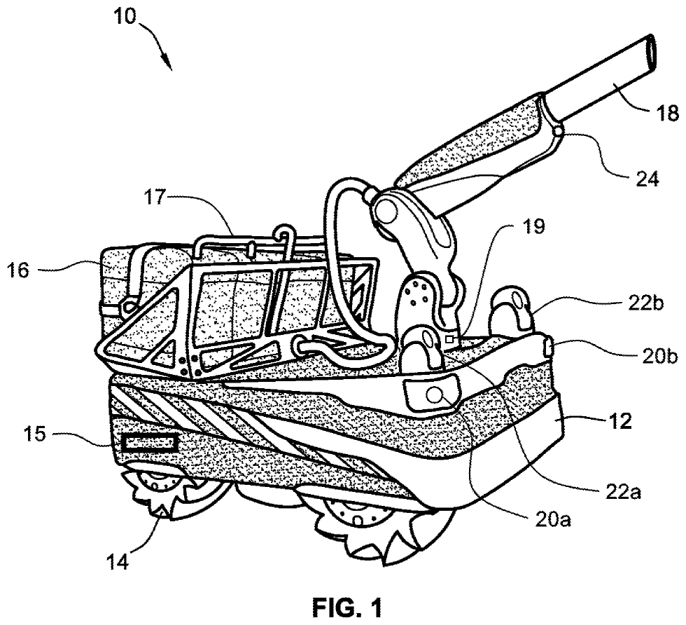

Autonomous vehicle 10 is shown in FIG. 1 including body structure 12 providing a mobility unit and four ground engaging wheels 14 (or alternatively, three wheels or a greater number of wheels or endless tracks). Mounted on the upper surface of body structure 12 is disinfecting agent tank 16 and articulating dispensing nozzle 18 (or an array of nozzles).

Autonomous vehicle 10 is intended to operate in an automated manner through various forms of guidance and programming. For example, vehicle 10 can receive global positioning system (GPS) signals which can provide approximate spatial position and orientation of the vehicle. Additional locating resources can be implemented including a suite of sensors such as cameras 22a, 22b, radar, ultrasound, lidar 18a, 18b, and other technologies familiar to those in the autonomous vehicle space. In addition, dedicated resources for a building structure may be used for example including embedded RFID tags, position beacons and signal grids can be provided for allowing vehicle 10 to follow a precise track. Guidance is controlled by an onboard electronic communication module (ECM) 15 incorporating a mobility module. In addition to locating vehicle 10 and allowing it to follow a desired course, a control function is provided for controlling dispensing nozzle 18, allowing it to be oriented in a desired manner and operated to dispense a disinfecting/decontamination material.

Preferably autonomous vehicle 10 is powered by onboard batteries and an electric motor driven drivetrain.

In a preferred embodiment, the ECM 15 uses a combination of at least two sensing systems, namely lidar sensors 20 and one or more articulating cameras 22a, 22b which faces outward, to control the vehicle 10. First, the vehicle 10 may be driven through a predetermined path or an algorithmic path while employing the lidar sensors 20 (or ultrasound, radar, floor facing cameras, ceiling facing cameras) to map a floorplan, i.e. a configuration of the space through which the robot can move and obstacles through which the robot cannot move. See, e.g. U.S. Pat. Nos. 5,999,866 and 6,459,955, the disclosures of which are incorporated by reference in their entirety. During the process of mapping the floor, the outward facing cameras 22 can be used to image the obstacles, and preferably also identify which obstacles represent objects to be cleaned (e.g. a row of seats in an airport). See, e.g. U.S. Pat. No. 5,378,969, the disclosure of which is incorporated by reference in its entirety. The data from the lidar sensors 20 and the cameras 22 can be coordinated so that the camera images can be used as a secondary confirmation of vehicle position.

Having mapped the floorplan, the ECM 15 can be programmed to follow a predetermined path through the floorplan. As alluded to above, the ECM 15 can also be programmed for special object identification, e.g. to identify particular objects based on the location or premises (particularly shaped chairs or benches in an airport, a desk within a school, etc.) that are to be cleaned or disinfected. While the ECM 15 may anticipate an object to be treated at certain points along the path based on the initial mapping, the cameras 22 is used during normal operation to identify objects for cleaning/disinfecting/decontaminating and corresponding control of the dispensing nozzle 18. Preferably, the cameras 22 have a common or substantially overlapping range of motion (x-y-z axes) as the articulating nozzle 18, both of which can be moved in three dimension in a coordinated or cooperating fashion.

One particular advantage attributed to use of autonomous vehicle 10 is its capability to provide data collection to validate area covered and disinfectant/decontamination material dispersal area. The cameras 22a, 22b may be used to confirm that a particular surface or object has been properly coated with disinfecting agent, for recording in the ECM 15. The cameras 22 may include filters or additional built-in sensors (e.g. infrared, ultraviolet, or other wavelength groups of the electromagnetic spectrum.) Alternately or in combination, a second camera system, such as a camera 24 directly mounted to the nozzle 18, may be employed for this function, leaving the first camera(s) 22 for object identification.

In a preferred embodiment of the present invention, a disinfecting agent is dispensed from nozzle 18 and supplied from tank 16 in an atomized manner. This could be done by a high-pressure feed system to a restricted flow nozzle. An electrostatic charging system 17 is located within or adjacent the tank 16 for creating an electrostatic charge in the atomized spray. The system 17 applies an electrostatic charge to the atomized disinfecting fluid, which has the effect of causing the atomized droplets of disinfecting fluid to be attracted to nearby surfaces. This effect is sufficient to enable finally dispersed droplets of the disinfecting solution to follow curved paths to attach to surfaces. This provides a more complete application of disinfecting fluid with a reduction in waste of “oversprayed” fluid dispensed into the air which does not deposit on the intended surfaces.

In an alternate embodiment, the single nozzle 18 is replaced with a multi-head sprayer array (not shown) to allow for large area coverage both while the vehicle is moving and when slowing or stopped.

When vehicle 10 is slowing or stopping, spray from the single or multihead sprayer array can be reduced or shut off to align with defined disinfectant/decontamination material dispersal areas needed to be covered. Even when vehicle 10 is running, flow from the single or multihead sprayer array can be reduced or shut off to align with defined disinfectant/decontamination material dispersal areas needed to be covered. For example, if a glass area does not need to be covered on one side, those facing spray heads can be shut down during that pass and then turned back on when coverage area applies. The ECM 15 is programmed to coordinate both motors for the nozzle 18, motors for the cameras 22, and motors for moving the vehicle 10 via its wheels 14 to both spray the entire identified object as well as confirm the status of the cleaning or disinfecting procedure. A separate controller 19 may also be provided for controlling the nozzle 18 and feed from the tank 16, and which is coordinated and in communication with the ECM 15.

1. An autonomous disinfectant/decontamination vehicle for applying a disinfectant/decontamination agent to surfaces of objects located at various places within a space, the vehicle comprising:
a frame having ground engaging wheels or tracks;
a dispensing nozzle rotatably attached relative to the frame, the dispensing nozzle configured tor spray a disinfectant/decontamination agent;
at least one controller for operating the wheels or tracks and the dispensing nozzle, the at least one controller having a floorplan and configured to enable the vehicle to follow a desired course within the floorplan;
a first sensing system including one or more sensors configured to sense objects using at least one of lidar, radar, camera and ultrasound, the first sensing system configured to provide floorplan data to the at least one controller;
a second sensing system including at least one camera facing laterally outwardly from the frame, the second sensing system configured to provide ;
the first sensing system configured to detect the presence of objects engaging the ground and cooperate with the at least one controller to move the vehicle along the desired course within the floorplan, the second sensing system configured to detect an identity and location of an object extending upwardly from the ground relative to the frame, whereby the at least on controller is configured to operate the dispensing nozzle to spray a disinfectant/decontamination agent onto surfaces of the object.

2. The vehicle of claim 1, wherein the dispensing nozzle is rotatable in three directions and controlled by the at least one controller in coordination with the second sensing system

3. The vehicle of claim 1, wherein the floorplan is derived from operating the within the space and mapping ground spaces through which the vehicle can and cannot move using the first sensing system.

4. The vehicle of claim 1, wherein the vehicle is movable along the desire course by a vehicle motor, and wherein the nozzle is rotatable relative to the frame via a nozzle motor, and wherein the camera is rotatable relative to the frame via a camera motor, and wherein the at least one controller coordinates the vehicle motor, nozzle motor and camera motor to identify objects to be disinfected as the vehicle is moving along the desired course and spray the disinfectant/decontamination agent onto surfaces of the object.

5. The vehicle of claim 4, wherein the at least one controller is further configured to confirm that the disinfectant/decontamination agent has been sprayed onto surfaces of the object using the second sensing system.

6. The vehicle of claim 1, wherein the at least one controller is further configured to confirm that the disinfectant/decontamination agent has been sprayed onto surfaces of the object using a third sensing system mounted for movement with the nozzle.

7. The vehicle of claim 6, wherein the third sensing system has sensors for detecting at least one of reflected light, infrared and ultrasound.

8. An method for applying a disinfectant/decontamination agent to surfaces of objects located at various places within a space, the method comprising:
providing an autonomous disinfectant/decontamination vehicle having a frame having ground engaging wheels or tracks, a dispensing nozzle rotatably attached relative to the frame and to spray the disinfectant/decontamination agent, at least one controller for operating the wheels or tracks and the dispensing nozzle, a first sensing system including one or more sensors configured to sense objects using at least one of lidar, radar, camera and ultrasound, and a second sensing system including at least one camera facing laterally outwardly from the frame,
moving the vehicle through the space while using the first sensing system to map a floorplan of the space that identifies areas of the space through which the vehicle can and cannot move;
determining a desired course through the space for applying a disinfectant/decontamination agent to surfaces of objects; and
operating the vehicle along the desired course and utilizing the second sensing system to identify objects to be disinfected or decontaminated and operate the dispensing nozzle to spray the disinfectant/decontamination agent on surfaces of the identified objects.

9. The method of claim 8, wherein the step of moving the vehicle through the space to map a floorplan include activating the second sensing system to identify preliminary objects within the space that are desired to be disinfected or decontaminated and recording the location of the preliminary objects.

10. The method of claim 8, wherein the step of operating the vehicle through the space includes comparing a location of a current object sensed by the second sensing system with the location of the preliminary objects.

11. The method of claim 8, wherein the first sensing system is configured to provide a location of the vehicle within the floorplan during the step of operating the vehicle.

12. The method of claim 8, wherein the first sensing system is configured to detect the presence of objects engaging the ground and cooperate with the at least one controller to move the vehicle along the desired course within the floorplan,

13. The method of claim 8, wherein the second sensing system is configured to detect an identity and location of an object extending upwardly from the ground relative to the frame, whereby the at least on controller is configured to operate the dispensing nozzle to spray a disinfectant/decontamination agent onto surfaces of the object.

14. The method of claim 8, wherein the vehicle is movable along the desire course by a vehicle motor, and wherein the nozzle is rotatable relative to the frame via a nozzle motor, and wherein the camera is rotatable relative to the frame via a camera motor, and wherein the at least one controller coordinates the vehicle motor, nozzle motor and camera motor to identify objects to be disinfected as the vehicle is moving along the desired course and spray the disinfectant/decontamination agent onto surfaces of the object.

15. The method of claim 14, wherein the at least one controller confirms that the disinfectant/decontamination agent has been sprayed onto surfaces of the object using the second sensing system.

16. The method of claim 14, wherein the at least one controller is further configured to confirm that the disinfectant/decontamination agent onto surfaces of the object using a third sensing system mounted for movement with the nozzle.

17. The method of claim 16, wherein the third sensing system has sensors for detecting at least one of reflected light, infrared and ultrasound.

18. An autonomous disinfectant/decontamination vehicle for applying a disinfectant/decontamination agent to surfaces comprising:
a mobility unit including a frame having ground engaging wheels or tracks;
a mobility controller for enabling the mobility unit to follow a desired course;
a dispensing nozzle for spraying an atomized spray of the disinfectant/decontamination agent;
an electrostatic charging system for creating an electrostatic charge in the atomized spray, and
a controller for operating the dispensing nozzle and the electrostatic charging system to apply the atomized spray to the surfaces.

19. The vehicle in accordance with claim 18, further comprising a plurality of the dispensing nozzles are provided in an array of dispensing nozzles.

20. The vehicle in accordance with claim 18, further comprising: a spray controller for activating the nozzle to apply the disinfectant/decontamination agent in a program manner to specific surfaces along a course controlled by the mobility controller.
